(19) United States
(12) Patent Application Publication
(10) Pub. No.: US 2022/0076833 A1
(43) Pub. Date: Mar. 10, 2022

(54) METHOD AND DEVICE FOR CREATING A CONTINUOUS COURSE AND FOR PREDICTING THE FLUID BALANCE OF A PATIENT

(71) Applicant: B. Braun Melsungen AG, Melsungen (DE)

(72) Inventors: Christian Baumgartner, Graz (AT); Katharina Bergmoser, Hitzendorf (AT)

(21) Appl. No.: 17/415,330

(22) PCT Filed: Dec. 18, 2019
(86) PCT No.: PCT/EP2019/085960

§ 371 Date: Jun. 17, 2021

(30) Foreign Application Priority Data
Dec. 20, 2018 (DE) 10 2018 009 902.5

(51) Int. Cl. G16H 50/20; G16H 20/40; G16H 40/63; G16H 20/17

(52) U.S. Cl. CPC G16H 50/20; G16H 20/17; G16H 40/63; G16H 20/40

(57) ABSTRACT

A method, device and program product for detecting the fluid balance of a patient in which a transfer function is established by which a continuous course of the fluid balance of the patient can be established for the past, and by which a continuous course of the fluid balance can be predicted for the future.

### CROSS-REFERENCE TO RELATED APPLICATION(S)

This application is the United States national phase entry of International Application No. PCT/EP2019/085960, filed Dec. 18, 2019, and claims the benefit of priority of German Application No. 10 2018 009 902.5, filed Dec. 20, 2018. The contents of International Application No. PCT/EP2019/085960 and German Application No. 10 2018 009 902.5 are incorporated by reference herein in their entireties.

### FIELD

The present invention relates to a method and a device for establishing a continuous course of the preferably cumulative fluid balance of a patient and/or of the preferably cumulative fluid intake to a patient in the past and for predicting a continuous course of the preferably cumulative fluid balance of the patient and/or of the preferably cumulative fluid intake to the patient in the future. Another aspect of the present invention relates to a program product. The term “cumulative” hereinafter shall be understood to be the “accumulation of all sources and sinks over time”.

### BACKGROUND

In up-to-date intensive care usually large quantities of fluids, e.g. in the form of drugs, nutrient solutions, saline solutions or liquids for extracorporeal blood treatment are supplied to patients. Usually, in the initial phase of their stay in the intensive care unit an excess of liquid is supplied to a patient.

In order to stabilize the patient hemodynamically in the long run, a balance must be found between the total quantity of the liquids supplied to the patient and the total quantity of the liquids lost by the patient. This is usually achieved within the scope of a fluid balancing of the patient by establishing a fluid balance of the patient by subtracting at a particular point in time the total quantity of the liquids lost by the patient from the total quantity of the liquids supplied to the patient at said point in time and by appropriately adapting the liquid supply.

In clinical practice, such fluid balance is usually collected every 24 hours during the patient's stay in the intensive care unit. The attending physician thus is provided with merely discrete values and, resp., individual data points of the collected fluid balance on the basis of which the further course of treatment (e.g. administration of drugs, administration of fluids etc.) must be decided. In addition, the attending physician usually obtains a relative fluid balance only which reproduces the variation of the fluid balance of the patient since the latest collection of the fluid balance (viz. e.g. 24 hours before).

The information obtained by the attending physician regarding the fluid balance of a patient and on the basis of which the further course of treatment has to be decided therefore reflects the fluid balance state of the patient frequently only very inaccurately and thus forms a suboptimal basis of decision for adapting the further course of treatment.

This increases the risk of a patient involved in a stay in intensive care.

### SUMMARY

Since an accurate collection and setting of the fluid balance of a patient as well as of the drug administration adapted thereto especially for critically ill patients in intensive care is an important factor for reducing the mortality, it is the object underlying the present invention to provide a method and a device which provides the attending physician with more accurate and more detailed information concerning the fluid balance of a patient and thus forms a more solid basis for deciding on the further course of treatment of the patient.

The afore-mentioned object is achieved by a method, a device and a program product. By means of the program product, existing devices such as blood treatment or fluid balancing systems can be retrofitted in accordance with the invention.

In the method for fluid balancing and, resp., detecting the fluid balance of a patient, not only individual data points of the fluid balance are collected, as compared to the state of the art, but for a patient a transfer function (also referred to as system function or controlled system) is established by means of which a preferably continuous course of the fluid balance of the patient (measured or calculated from measuring values) can be reproduced for the past (afterwards) and a predicted preferably continuous course of the fluid balance (and, resp., a predicted trajectory of the trend of the fluid balance) can be established for the future.

A transfer function used within the scope of the invention may be, for example, a time-discrete transfer function P[z] of the general formula reproduced below:

$$P [ z ] = \frac{Y [ z ]}{U [ z ]} = \frac{{b}_{m} {z}^{m} + {b}_{m - 1} {z}^{m - 1} + … + {b}_{1} z + {b}_{0}}{{a}_{n} {z}^{n} + {a}_{n - 1} {z}^{n - 1} + … + {a}_{1} z + {a}_{0}}$$

Here Y[z] and U[z] are the Z transformed values of output and input variables. b₀ to bₘ and a₀ to aₙ are the coefficients of the transfer function. In addition, n denotes the degree of the transfer function.

It is the central idea of the invention that:
from a course or trend of a cumulative fluid intake (CFI) (measured or provided by an approximation procedure), and
from a course or trend of a cumulative fluid balance (CFB) which was/is calculated from the course or trend of the cumulative fluid intake (measured or provided by an approximation procedure) by subtraction of all cumulative fluid losses which are/have been occurring,
   a transfer function for reproducing the course or trend of the cumulative fluid balance is developed. A future course or trend of the cumulative fluid intake forecast by an approximation procedure then is incorporated in the transfer function so as to establish a predicted continuous course or trend of the cumulative fluid balance of the patient for the future.

That is to say, the course of the CFI for the past is preferably established only by means of the approximation procedure. The course of the CFB for the past is established by using the CFI preferably approximated by means of approximation procedures as input for the transfer function. The output sequence then includes the approximated CFB. The CFI is predicted by extrapolating the CFI preferably approximated by means of the approximation procedure via the gradient and, resp., a target CFI can also be predicted by integrating the transfer function into a control loop and setting a target CFB.

The CFB is predicted by using the CFI preferably approximated and extrapolated via the approximation procedure as input for the transfer function. The output sequence then includes the approximated predicted CFB and, resp., the CFB can also be predicted by integrating the transfer function into a control loop and by defining a target CFB. The output of the control loop then includes the target CFB to be actually expected which corresponds to the target CFB, if the control loop succeeds in finding a matching target CFI course.

In other words, the transfer function in combination with the approximation procedure serves as a mathematical model by means of which from detected data, for example concerning the liquid supply to the patient and the liquid lost by the patient, both a continuous course or trend of the fluid balance and/or of the fluid intake of the patient can be reproduced for the past and a continuous course or trend of the fluid balance and/or of the fluid intake of the patient can be predicted for the future.

Accordingly, by means of the transfer function preferably a cumulative fluid balance is depicted which, in contrast to the relative fluid balance known from the state of the art which indicates only the difference from the latest collected data point of the fluid balance, reproduces the total course of the fluid balance for the patient since the beginning of recording (for example since admission of the patient to the intensive care unit).

The preferably cumulative fluid balance is calculated by subtracting the total quantity of the fluids lost by the patient from the total quantity of the fluid intake to the patient. Of preference, the total quantity of the liquids lost by the patient (fluid losses) is subtracted from the total quantity of the fluid/liquid supply to the patient.

Accordingly, drugs, nutrient solutions, saline solutions etc. administered to the patient and other liquids supplied to the patient in any way such as via stomach tubes, intra-pulmonically, intra-nasally etc. are included in the total quantity of the fluid/liquid supply to the patient.

When determining the total quantity of the liquids lost by the patient, all liquids lost by the patient such as the quantity of urine excreted by the patient, insensible losses, but also the fluids lost e.g. via wound drains or pulmonary suctions are included.

Alternatively or additionally to the cumulative fluid balance, by means of an approximation procedure also a cumulative fluid intake can be depicted which reproduces the total course of the fluid intake to the patient since the beginning of recording (for example since the admission of the patient to intensive care).

Apart from the fluid intake and the fluid balance, also other data relating to the patient can be taken into consideration. For example, effects or side-effects of various drugs administered to the patient on the fluid balance or liquid balance of the patient can be considered. Likewise, physiological data of the patient, blood values of the patient or else information concerning the patient's age, sex, possible prior diseases, a scheduled course of treatment including scheduled operations etc. can be taken into consideration when e.g. embedding the transfer function into a control loop.

According to one aspect of the invention, a method according to the invention comprises the following steps of:

detecting and/or determining data concerning the cumulative fluid intake (CFI) to a patient and the cumulative fluid balance (CFB) of the patient;

establishing a transfer function for said patient based on the detected and/or determined data concerning the cumulative fluid intake and the cumulative fluid balance, wherein by means of the transfer function in combination with an approximation procedure the cumulative fluid intake and/or the cumulative fluid balance of the patient can be established or reproduced from the data concerning the cumulative fluid intake and the cumulative fluid balance, and

calculating, by means of the transfer function in combination with the approximation procedure, a predicted continuous course or trend of the cumulative fluid intake and/or of the cumulative fluid balance of the patient for the future.

For example, in an event in which the course of the cumulative fluid intake to a patient is to be depicted by means of an approximation procedure, the method can comprise the following steps of:

detecting data concerning the cumulative fluid intake to a patient;

applying an approximation procedure for said patient on the basis of the detected data concerning the cumulative fluid intake, wherein the cumulative fluid intake of the patient can be established by means of the approximation procedure from the detected data concerning the cumulative fluid intake, and

calculating a continuous course or trend of the cumulative fluid intake of the patient for the past as well as a predicted continuous course of trend of the cumulative fluid intake of the patient for the future.

For example, in an event in which the course of the cumulative fluid balance to a patient is to be depicted by means of the transfer function, the method can comprise the following steps of:

detecting data concerning the cumulative fluid intake and the cumulative fluid balance of a patient;

establishing a transfer function for said patient on the basis of the detected data concerning the cumulative fluid intake and the cumulative fluid balance, wherein the cumulative fluid balance of the patient can be established by means of the transfer function in combination with an approximation procedure from the data concerning the cumulative fluid intake and the cumulative fluid balance (in order to obtain the CFB course, either the raw CFI data or the approximated CFI calculated via the approximation procedure can be used as input for the transfer function. By using the approximated CFI, a CFB trajectory and no unsmoothed CFB course is obtained), and

calculating, by means of the transfer function in combination with an approximation procedure, a continuous course or trend of the cumulative fluid balance of the patient for the past as well as a predicted continuous course or trend of the cumulative fluid balance of the patient for the future.

In addition, from the course of the cumulative fluid intake the course of the cumulative fluid balance can be established by means of a transfer function in combination with an approximation procedure. In such event, a method according to the invention comprises the steps of:

detecting data concerning the cumulative fluid balance and the cumulative fluid intake to a patient;

establishing a transfer function for said patient on the basis of the detected data concerning the cumulative fluid intake and the cumulative fluid balance, wherein the cumulative fluid balance of the patient can be established by means of the transfer function in combination with the approximation procedure from the data concerning the cumulative fluid intake, and

calculating, by means of the transfer function in combination with the approximation procedure, a continuous course or trend of the cumulative fluid balance of the patient for the past as well as a predicted continuous course or trend of the cumulative fluid balance of the patient for the future.

For example, the established continuous histories of the cumulative fluid intake and/or the cumulative fluid balance for each patient for the past and for the future can be displayed jointly or separately on a display device.

The continuous histories of the cumulative fluid intake and/or the cumulative fluid balance for each patient for the past and the future displayed in this way thus can be made available to an attending physician when deciding on the further course of treatment. Said physician thus is provided not only with individual data points but with a continuous course of the cumulative fluid intake and/or the cumulative fluid balance for each patient during his/her entire stay in the intensive care unit as well as with a prediction of the further development of the course of the cumulative fluid intake and/or the cumulative fluid balance for said patient. This will improve the basis for decision of the attending physician when determining the further course of treatment and thus will increase the safety of the patient.

In accordance with another aspect of the invention, a method according to the invention further comprises the following steps of:

setting a target value and, resp., a target course of the cumulative fluid balance of the patient; and

calculating a course of the cumulative fluid intake to the patient by means of which an actual value and, resp., an actual course of the cumulative fluid balance of the patient can be brought in line with the set target value and, resp., target course of the cumulative fluid balance.

Then fluid is supplied to the patient on the basis of the calculated course of the cumulative fluid intake to the patient by means of which the actual value and, resp., the actual course of the cumulative fluid balance for the patient can be brought in line with the set target value and, resp., target course of the cumulative fluid balance, until the actual value and, resp., the actual course of the cumulative fluid balance for the patient corresponds to the set target value and, resp., target course of the cumulative fluid balance. The target value and, resp., target course may correspond to a desired positive or negative gradient of the course within a particular time interval.

Said aspect of the invention offers the advantage that the cumulative fluid intake can be controlled on the basis of the transfer function determined for a patient semi-automatically or else fully automatically such that an actual value and, resp., actual course of the cumulative fluid balance for the patient is adjusted to the set target value and, resp., target course of the cumulative fluid balance. Resorting to the continuous course of the cumulative fluid balance predicted by means of the transfer function in this case permits a more accurate setting of the cumulative fluid intake than this is possible by the individual data points of the fluid balance known from the state of the art.

Also, the cumulative fluid intake to the patient can be controlled by resorting to the transfer function for said patient. In this case, the method comprises the steps of:

setting a target value and, resp., target course of the cumulative fluid intake to the patient; and

calculating a course of the cumulative fluid intake to the patient by means of which an actual value and, resp., actual course of the cumulative fluid intake for the patient can be brought in line with the set target value and, resp., target course of the cumulative fluid intake.

Then fluid is supplied to the patient on the basis of the calculated course of the cumulative fluid intake to the patient by means of which the actual value and, resp., actual course of the cumulative fluid intake for the patient can be brought in line with the set target value and, resp., target course of the cumulative fluid intake, until the actual value and, resp., the actual course of the cumulative fluid intake for the patient corresponds to the set target value and, resp., target course of the cumulative fluid intake. The target value and, resp., target course may correspond to a desired (positive) gradient of the course within a particular time interval.

According to another aspect of the invention, a method according to the invention comprises a step of determining a turning point by means of an approximation procedure in the course of the cumulative fluid balance and/or the cumulative fluid intake on the basis of the course of the cumulative fluid balance and/or the cumulative fluid intake for the past as well as of the predicted continuous course of the cumulative fluid balance and/or the cumulative fluid intake for the future.

The turning point can be determined either from the raw data for CFI or CFB for the past (this is done here for the CFI to obtain the CFI approximation) or from the predicted data for the CFI (data are formed either by approximation and extrapolation or when setting a target CFB course in the control loop) or the predicted data for the CFB (data are formed either by applying the transfer function to the CFI smoothed and extrapolated with the aid of the approximation procedure or via the control loop output).

The turning point shows the point in the course of the cumulative fluid balance and/or the cumulative fluid intake in which the patient passes from the Ebb phase (acute phase) of the shock with a reduced metabolism to the Flow phase (secondary phase) of the shock.

For example, a method according to the invention may comprise a step of determining a turning point in the course of the cumulative fluid intake on the basis of the continuous course of the cumulative fluid intake for the past as well as on the basis of the continuous course of the cumulative fluid intake for the future predicted by means of approximation procedures or in combination with the transfer function.

Alternatively or additionally, a method according to the invention may comprise a step of determining a turning point in the course of the cumulative fluid balance on the basis of the course of the cumulative fluid balance or on the basis of the continuous course of the cumulative fluid balance for the past calculated by means of the transfer function in combination with the approximation procedure as well as of the predicted continuous course of the cumulative fluid balance for the future.

Accordingly, the turning point of the cumulative fluid balance and/or the cumulative fluid intake can be determined by means of an approximation procedure in which the potential turning point closest to an actual turning point is selected from a plurality of potential turning points in the course of the cumulative fluid balance and/or the cumulative fluid intake of the patient.

For a particular time interval, for example the duration of the patient's stay in the intensive care unit, e.g. each point in time can be examined as potential turning point. Within the entire particular time interval, the established values can be examined as potential turning points even at fixed subordinate time intervals, however. For example, during the patient's stay in the intensive care unit an examination for establishing the turning point or for examining a potential turning point can be carried out every hour. Other fixed subordinate time intervals may be set at will.

When a potential turning point is examined in a particular time interval, e.g. from t₀ to t₁, the potential turning point is preferably defined such that the time interval from t₀ to t₁ is divided into two segments by the potential turning point. Then, for example by means of linear interpolation as well as linear regression (both segments are approximated via linear regressions. The straight line 1 describes the trajectory from t₀ to the (potential) turning point, the straight line 2 describes the trajectory from the (potential) turning point to t₁. An extrapolation takes place only with the prediction of the CFI trajectory from the point in time t₁ to t₂ (time of prediction). Between t₀ and t₁ no extrapolation takes place, as all data are given. An interpolation may be required when data points are missing in the previous course of CFI), the course of the cumulative fluid balance and/or the cumulative fluid intake is established between the point in time t₀ and the potential turning point in the first segment of the time interval from t₀ to t₁ and between the potential turning point and the point in time t₁ in the second segment of the time interval from t₀ to t₁. In the potential turning point thus the two straight lines are intersecting each of which reproduces the course of the cumulative fluid balance and/or of the cumulative fluid intake in the first segment and in the second segment of the time interval from t₀ to t₁.

Instead of a linear interpolation and, resp., a linear regression, basically also any other function, e.g. a non-linear function, may be used for interpolation and, resp., regression. In addition, the turning point can be determined by approximation for any number of passes and, resp., at any number of iterations.

In order to establish the quality of determining the turning point by approximation, i.e. to determine how close an examined potential turning point comes to the actual turning point, the statistic deviation between the course approximated by means of preferably linear regression by the two straight lines and the actual course is established. Preferably, for this purpose the Root-Mean-Squared-Error (RMSE) is considered. For each potential turning point the calculated RMSE is compared to the previously lowest RMSE of another potential turning point from any one of the preceding passes of the approximation procedure.

When the currently calculated RMSE value is lower, i.e. when the currently examined potential turning point comes closer to the actual turning point, the currently examined potential turning point is stored as the currently best one (i.e. the turning point closest to the actual turning point). Otherwise, the currently examined potential turning point is dismissed and the other potential turning point having the lower RMSE value from one of the preceding passes of the approximation procedure is stored as the turning point closest to the actual turning point.

The established closest turning point can also be displayed to an attending physician, for example by means of a display device, thus further improving the basis of information on the basis of which a decision must be taken about the further course of treatment.

The gradient of the straight line across the potential turning point which reproduces the course of the cumulative fluid intake to the patient in the second segment of the time interval from t₀ to t₁ is used, for example within the scope of linear extrapolation, to predict the cumulative fluid intake to the patient, e.g. in a second time interval from t₁ to t₂ (prediction interval).

In combination with the transfer function, on the basis of the gradient of the straight line which reproduces the course of the cumulative fluid intake to the patient in the second segment of the time interval from t₀ to t₁, the course of the cumulative fluid balance for the patient can be predicted in the second time interval from t₁ to t₂.

According to one aspect of the invention, the transfer function for the patient is periodically updated and, resp., adapted at predetermined time intervals, for example every hour, on the basis of newly collected data concerning the preferably cumulative fluid balance and fluid intake. The adaptation can take place, for example, for the duration of the entire stay of the patient in the intensive care unit. This helps ensure that the transfer function reflects the cumulative fluid balance of the patient and, resp., the cumulative fluid intake to the patient as accurately as possible for the duration of the entire stay of the patient in the intensive care unit.

According to another aspect of the invention, the transfer function for the patient is set on the basis of patient cohort-specific information in which a respective particular patient cohort is linked with a particular transfer function to be applied to said patient cohort.

Accordingly, the patient cohort-specific information is preferably produced by identifying and, resp., extracting joint features of the transfer function established for a particular patient cohort in the past, such as e.g. poles, zeros, stability, pulse response, step response etc., especially by means of statistic methods, artificial intelligence, neuronal networks, machine-learning or logistic regression and taking the features identified in this way as a basis when the transfer function for a patient belonging to said particular patient cohort is established.

A particular patient cohort here denotes a group of patients having particular features in common. Said features may be, for example, the age, the sex, a treatment or operation to be made, possible prior diseases, the time of stay in intensive care, laboratory parameters etc. Patient cohorts may be formed on the basis of any common features.

With establishing the transfer function for the patient on the basis of patient cohort-specific information, it is thus not necessary to individually establish a particular transfer function for a particular patient from the data of the cumulative fluid intake and the cumulative fluid balance established for said patient, but the common features of the transfer functions established for a particular patient cohort in the past can be resorted to as empirical values.

Establishing such transfer function for a patient on the basis of patient cohort-specific information may reduce the time required for computing, as no individual transfer function must be established.

Alternatively or additionally, a transfer function for the patient on the basis of patient cohort-specific information may also serve for checking a transfer function individually established for said patient for plausibility by comparing the individually established transfer function to a transfer function established on the basis of patient cohort-specific information. Such comparison increases the accuracy of the transfer function and thus further increases the patient safety.

A method according to the invention can either be applied to one single patient only or it can be applied also to a plurality of patients. When the method is applied to a plurality of patients, for each patient among the plurality of patients a respective transfer function can be individually established. As an alternative or in addition to this, patient cohorts can be formed also from the plurality of patients and, on the basis of cohort-specific information, transfer functions can be formed as described before.

The data flows of the data collected for the patients among the plurality of patients (e.g. concerning the fluid balance or the fluid intake) thus can be processed separately for each patient or else can be combined so that data pools are formed.

Another aspect of the invention relates to a device which is designed for implementing a method according to the invention as set forth by one of the foregoing aspects.

Preferably, the device in this case is a device for extracorporeal blood treatment, a drug administration system, a fluid management system or an infusion pump or any other device which is directly or indirectly related with the fluid balance or the liquid balance of the patient.

Such device preferably includes an element or means for collecting the data concerning the preferably cumulative fluid intake to the patient and/or concerning the preferably cumulative fluid balance of the patient.

Further, such device preferably includes a display device, for example a monitor/display by means of which the established information (histories, prediction values, turning points) can be made available to an attending physician.

Another aspect of the invention relates to a program product which causes a device to implement a method according to the invention in accordance with a foregoing aspect.

By means of such program product existing devices, such as e.g. a device for extracorporeal blood treatment, a drug administration system, a fluid management system, an infusion pump or any other device which is directly or indirectly related to the fluid balance or liquid balance of the patient, can be retrofitted. Such program product can be stored in a storage medium, for example.

### BRIEF DESCRIPTION OF THE DRAWING FIGURES

Hereinafter, an embodiment of the present invention will be exemplified in detail with reference to the figures.

FIGS. 1a to 1e exemplify the various steps of a method according to an embodiment of the invention.

FIGS. 2a to 2e exemplify the various steps of an approximation procedure for determining the trajectory of the cumulative liquid supply and simultaneously determining a turning point in the course of the cumulative fluid intake to a patient.

### DETAILED DESCRIPTION

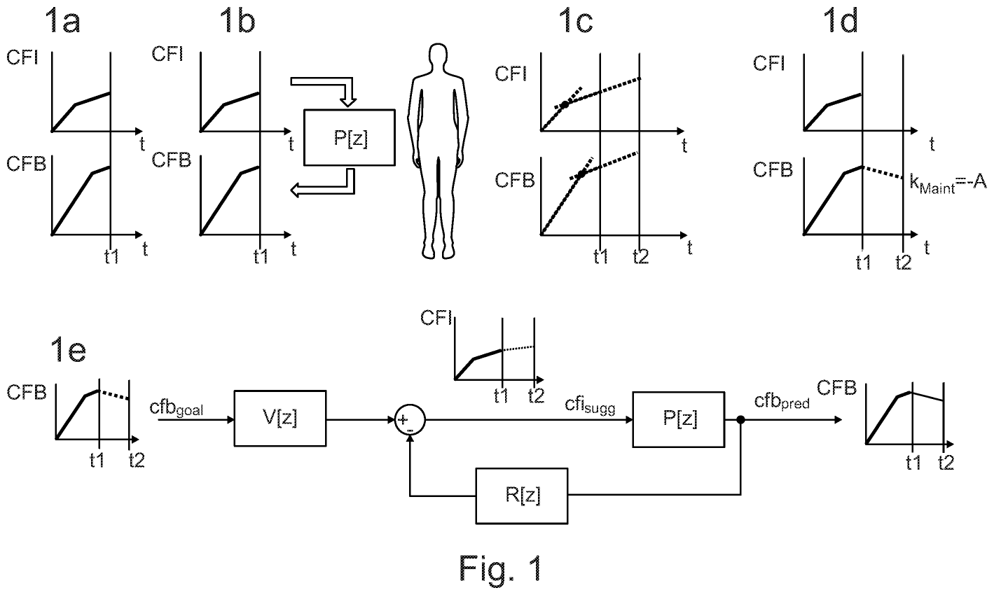

FIGS. 1a to 1e exemplify the various steps of a method according to an embodiment of the invention in which the method according to the invention is applied to a patient.

As illustrated in FIG. 1a, initially for a first time interval from t0 (origin) to t1 data concerning the cumulative fluid intake CFI to the patient and the cumulative fluid balance CFB of the patient are collected. The time interval from t0 to t1 may be defined at will and may comprise e.g. several minutes, one hour or even several hours such as 6 hours or 12 hours. The time interval may also comprise the entire duration of the stay of the patient in intensive care, for example several days.

Then, as shown in FIG. 1b, on the basis of the collected data a preferably time-discrete transfer function P[z] is established which reproduces a relation between the data concerning the cumulative fluid intake CFI to the patient and the cumulative fluid balance CFB of the patient.

Accordingly, the course of the cumulative fluid intake CFI to the patient, in the present example since his/her admission to the intensive care unit, is used as input sequence of the transfer function, and the course of the cumulative fluid balance CFB of the patient, in the present example since his/her admission to the intensive care unit, is used as output sequence of the transfer function.

The transfer function P[z] here describes a relation between the cumulative fluid intake CFI to the patient and the cumulative fluid balance CFB of the patient (i.e. the transfer function indicates how CFB is varying in response to CFI). When the data of the cumulative fluid intake CFI or the data of the cumulative fluid intake CFI approximated by means of approximation procedures are obtained as input sequence as well as the transfer function P[z], the dedicated values of the cumulative fluid balance CFB thus can be calculated.

As illustrated in FIG. 1c, based on the course of CFI and CFB established on the basis of the collected data concerning CFI and CFB of the patient in the time interval from t0 to t1, then a prediction is made for the further course of CFI and CFB in a future time interval from t1 to t2.

Accordingly, at first the turning point in the course of CFI is established by means of the afore-described approximation procedure (to obtain the CFI approximated so far) by stepwise examining points along the course as potential turning points and selecting the potential turning point coming closest to the actual turning point (i.e. for example the turning point having the lowest RMSE value with respect to the actual turning point).

In the approximation procedure, a first straight line which reproduces the course of CFB and, resp., CFI for the segment between t0 (origin) and the potential turning point is laid across the origin and the potential turning point. Then, by means of another linear regression, a second straight line which reproduces the course of CFB and, resp., CFI for the segment between the potential turning point and t1 is laid across the turning point.

Said second straight line, especially the gradient of the second straight line, can be used, as shown in FIG. 1c, for predicting the further course of CFB and, resp., CFI for the future, in this example for a time interval from t1 to t2 exceeding the point in time t1.

The diagrams shown in FIG. 1c correspond by way of example to the data or histories of the cumulative fluid intake and, resp., fluid balance of the patient displayed on a display device to an attending physician.

As shown in FIG. 1d, the attending physician may fix a target value or a target course of CFI or CFB for the patient. In the example of FIG. 1d, a target course of the cumulative fluid balance CFB was fixed for the time interval from t1 to t2. Accordingly, a value k_{Maint} is set which reflects a desired gradient of the course of CFB for the time interval from t1 to t2. In FIG. 1d, a value of (−A) was set which reflects a desired gradient, negative in this example, of the course of CFB for the time interval from t1 to t2.

As illustrated in FIG. 1e, then a cumulative target fluid balance and, resp., a target course of the cumulative fluid balance cfb_{goal} is established on the basis of the target course of the cumulative fluid balance CFB. Corresponding to said target course of the cumulative fluid balance CFB, by means of embedding the transfer function P[z] into a control loop in the form of an inverse transfer function V(z), a suggestion for a course of the fluid intake to the patient cfi_{sugg} is determined for which the control loop predicts that with such course of the fluid intake to the patient the predicted course of the fluid balance cfb_{pred} corresponds to the set target course of the cumulative fluid balance CFB cfb_{goal}.

By means of a control loop R[z] the suggestion for the course of the cumulative fluid intake to the patient cfi_{sugg} is adapted so that the suggestion for the course of the cumulative fluid intake to the patient cfi_{sugg} corresponds exactly to the course of the cumulative fluid intake which, according to the transfer function P[z], results in the predicted course of the fluid balance cfb_{pred} coinciding with the target course of the cumulative fluid balance cfb_{goal}.

After that, fluid having the calculated course of the cumulative fluid intake cfi_{sugg} is supplied to the patient so that the desired course of the cumulative fluid balance CFB is obtained.

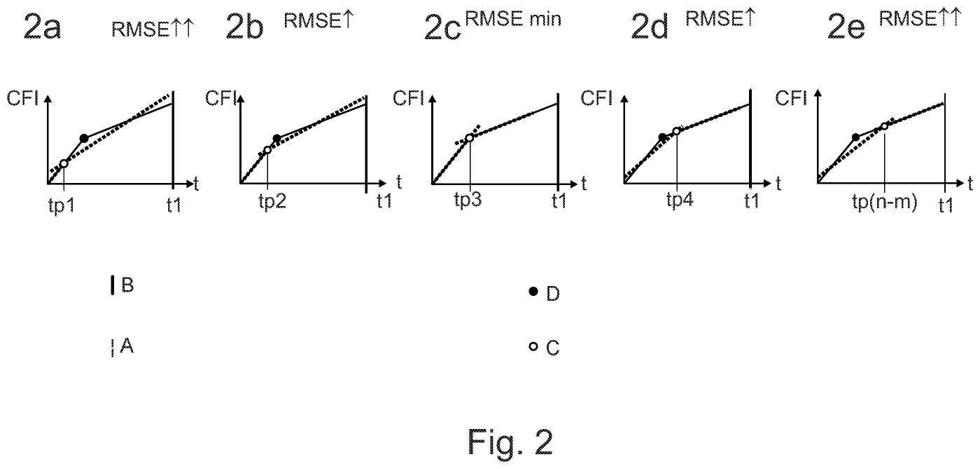

FIGS. 2a to 2e exemplify the various steps of an approximation procedure for determining the trajectory of the cumulative fluid intake CFI and simultaneously determining a turning point in the course of the cumulative fluid intake CFI to a patient. Alternatively or additionally, the trajectory including the turning point can also be determined in the course of the cumulative fluid balance CFB of the patient.

As illustrated in FIGS. 2a to 2e, by means of a linear regression and, resp., interpolation (A) the course of the cumulative fluid intake CFI (B) to a patient is reproduced at a time interval from t0 (origin) to t1. Different points along the course are examined as potential turning points (C).

In FIGS. 2a to 2e, the potential turning points tp1, tp2, tp3, tp4 and tp(n−m) are exemplified in which each of the two straight lines of the linear regression/interpolation of the course intersects in the first and second segments of the time interval from t0 to t1.

For each potential turning point the deviation of the approximated cumulative fluid intake from the actual cumulative fluid intake is established. Concretely, in this embodiment the deviation is established by establishing a Root-Mean-Squared-Error value (RMSE) which indicates the deviation of the respective approximated course from the actual course. The smaller the RMSE value, the closer the potential turning point comes to the actual turning point (D).

In the embodiment shown in FIGS. 2a to 2e, the potential turning point tp3 shown in FIG. 2c corresponds to the actual turning point and therefore has the lowest RMSE value (RMSE min).

In the approximation procedure according to the invention, the potential turning point having the lowest RMSE value is selected step by step. For example, at first the potential turning point tp1 shown in FIG. 2a is examined. As shown in FIG. 2a, tp1 is not very close to the actual turning point, however, and the dedicated RMSE value is correspondingly high (RMSE^{↑↑}). The potential turning point tp1 as well as the dedicated RMSE value and the dedicated straight lines of the linear regression/interpolation are then stored.

In the next pass of the approximation procedure, the potential turning point tp2 shown in FIG. 2b is examined. As shown in FIG. 2b, tp2 is closer to the actual turning point than the potential turning point tp1, and the RMSE value dedicated to the potential turning point tp2 is respectively lower (RMSE_{↑}) than the RMSE value (RMSE_{↑↑}) dedicated to the potential turning point tp1. The potential turning point tp2 as well as the dedicated RMSE value and the dedicated straight lines of the linear regression/interpolation then are stored and the potential turning point tp1 is dismissed.

For the potential turning points tp1 and tp2 shown in FIGS. 2a and 2b, the determination by approximation of the potential turning point tp3 was carried out by stepwise shifting of the potential turning point to be examined in the positive direction of the x axis (in FIGS. 2a and 2b starting from the origin to the right).

As illustrated in FIGS. 2d and 2e, the determination by approximation of the potential turning point tp3 can be carried out alternatively or additionally also by stepwise shifting of the potential turning point to be examined in the opposite direction of the x axis (in FIGS. 2d and 2e along the x axis toward the origin). In the model, at a particular interval starting from t0 each point in the course is tested as a potential turning point, until the end of the course (t1) is reached. If the best turning point has been found somewhere in the middle (which you do not know at that point in time), the RMSE will increase in the next steps, to be sure, but still the entire time sequence has to passed up to the end (t1) so that a possibly even lower RMSE value will not be missed. The examination is carried out from the first step to the last step starting from the origin to the right.

The method could also be started at t1 and could be terminated at t0. Then the method would not change the direction in the middle, either, but would move toward the origin in each step along the x axis.

1.-10. (canceled)

11. A method for fluid balancing at least one patient comprising the steps of:
detecting data concerning a cumulative fluid intake to the at least one patient and a cumulative fluid balance of the at least one patient;
establishing a transfer function for the at least one patient based on detected data concerning the cumulative fluid intake and the cumulative fluid balance, wherein by the transfer function in combination with an approximation procedure, the cumulative fluid intake and/or the cumulative fluid balance of the at least one patient is established from data concerning the cumulative fluid intake and/or the cumulative fluid balance; and
calculating, by the transfer function in combination with the approximation procedure, a past continuous course of the cumulative fluid intake and/or the cumulative fluid balance of the at least one patient for a past period of time and a predicted continuous course of the cumulative fluid intake and/or the cumulative fluid balance of the at least one patient for a future period of time,
wherein, based on the past continuous course and the predicted continuous course, a turning point of the cumulative fluid balance and/or of the cumulative fluid intake is determined at which the at least one patient passes from an Ebb phase of shock into a Flow phase of shock.

12. The method according to claim 11, wherein a cumulative fluid intake of the at least one patient for a future period of time is calculated by extrapolating the cumulative fluid intake approximated by the approximation procedure via a gradient, or the cumulative fluid intake of the at least one patient for the future period of time is calculated by integrating the transfer function into a control loop and setting a target value or target course of the cumulative fluid balance, and
wherein the cumulative fluid balance of the at least one patient for the future is calculated by using the cumulative fluid intake approximated and extrapolated via the approximation procedure as input for the transfer function, or the cumulative fluid balance of the at least one patient for the future is defined by the target value or target course of the cumulative fluid balance.

13. The method according to claim 11, further comprising the steps of:
setting a target value or target course of the cumulative fluid balance for the at least one patient; and
calculating a course of the cumulative fluid intake to the at least one patient by which an actual value or an actual course of the cumulative fluid balance for the at least one patient is brought in line with the set target value or target course of the cumulative fluid balance.

14. The method according to claim 11, wherein the turning point of the cumulative fluid balance and/or the cumulative fluid intake is determined by a turning point approximation procedure in which a potential turning point coming closest to an actual turning point is iteratively selected from a plurality of potential turning points.

15. The method according to claim 11, wherein the transfer function for the at least one patient is updated periodically at predetermined time intervals.

16. The method according to claim 11, wherein the transfer function for the at least one patient is established based on patient cohort-specific information in which a respective particular patient cohort is linked with a particular transfer function to be applied to each patient of said patient cohort.

17. The method according to claim 16, wherein the patient cohort-specific information is produced by common features of transfer functions established in a past period of time for a particular patient cohort being identified by statistical methods, artificial intelligence, neuronal networks, machine learning or logistic regression and by features identified in this way being taken as a basis when the transfer function for a patient belonging to said particular patient cohort is established.

18. A device configured for implementing the method according to claim 11.

19. The device according to claim 18, wherein said device is a device for extracorporeal blood treatment, a drug administration system, a fluid management system or an infusion pump or any other device which is directly or indirectly related to the cumulative fluid balance of the at least one patient.

20. A program product which causes a device to carry out the method according to claim 11.
